(19) United States
(12) Patent Application Publication
(10) Pub. No.: US 2012/0143048 A1
(43) Pub. Date: Jun. 7, 2012

(54) SURGICAL APPARATUS, EQUIPMENT AND METHODS

(75) Inventor: Patrick Finlay, Beaconsfield (GB)

(73) Assignee: MEDI MATON LIMITED, Beaconsfield, Buckinghamshire (DE)

(21) Appl. No.: 13/386,636

(22) PCT Filed: Jul. 22, 2010
(86) PCT No.: PCT/GB10/51212

§ 371 Date: Feb. 15, 2012

(30) Foreign Application Priority Data
Jul. 23, 2009 (GB) 0912824.0

(51) Int. Cl. A61B 5/05; A61B 19/00

(52) U.S. Cl. 600/424; 606/130

(57) ABSTRACT

A surgical apparatus comprising: a base unit; an arm coupled to the base unit, the arm comprising a first section and a second section which are moveable with respect to each other about a joint, the arm being configured to receive a guide-tube; a tracking un it coupled to the joint and configured to track movement of the first section of the arm with respect to the second section of the arm; and a processor configured to: receive intra-operative image data relating to the guide-tube and the anatomy of a patient from which an initial position and orientation of the guide-tube relative to the anatomy of the patient can be determined, the image data including image data relating to a target location within the patient, receive tracking information from the tracking unit, and track the position and orientation of the guide-tube based on the initial position and orientation and the tracking information such that the guide-tube may be aligned with respect to the target location within the patient using the tracked position and orientation of the guide-tube to allow the delivery of a surgical instrument to the target location.

### DESCRIPTION OF INVENTION

The present invention relates to a surgical apparatus, an orthopaedic surgical apparatus, a method operating a surgical apparatus, a method of operating an orthopaedic surgical apparatus, a guide-tube, and a method of forming a guide-tube.

Many surgical operations and medical procedures require a needle or other slender instrument to be guided into a patient to a target location which is not externally visible to the needle operator.

Conventionally, a practitioner (e.g. a surgeon) uses real-time fluoroscopic images of the patient to guide insertion of the needle to the target location. This is a difficult process requiring considerable skill and practice. The fluoroscopic images which are presented to the surgeon are only two dimensional images through an entire depth of the patient. Thus, although the needle may appear to be advancing correctly to the target location in a fluoroscopic image through single axis, the needle may be advancing considerably off-course in the direction of the axis of the fluoroscopic image. The practitioner must, therefore, continually adjust the axis of the fluoroscope at the same time as inserting the needle in order to ensure that the needle is being advanced correctly towards the target location.

The difficulty in the insertion of a needle in this manner increases the duration of the operation or procedure. As the operation or procedure is conventionally conducted using a fluoroscope, the longer the duration of the operation or procedure the greater the level of exposure of x-ray radiation for the patient, the practitioner and any assistants present during the operation or procedure. Long operations can also consume valuable resources and can be detrimental to the health of the patient. In orthopaedic surgery in particular a needle which has been incorrectly inserted into a bone (at an incorrect trajectory—for example) can weaken the bone significantly.

Surgical robotic assistants have been developed in an attempt to increase the speed and consistency of surgical operations which involve procedures which are time consuming and/or require considerable skill and practice to master. Such robotic assistants have traditionally been large and expensive devices.

There is, therefore, a need to provide a less cumbersome and complex surgical apparatus which can assist a surgeon or other practitioner or user in the insertion of a needle or other surgical instrument into a patient to a target location which is not externally visible.

Embodiments of the present invention seek to ameliorate some of the problems associated with the prior art.

One aspect of the present invention provides a guide-tube comprising: an elongate tubular main body having a cavity configured to receive a needle or other slender instrument; a plurality of markers disposed along a length of the tubular main body, the markers being opaque to a first imaging signal, such that the position and orientation of the guide-tube in a frame of reference can be substantially unambiguously determined by identifying the location of each of the plurality of markers in the frame of reference using the first imaging signal wherein the elongate tubular main body comprises two sections joined together at respective interface surfaces and one or more of the plurality of markers are arranged in respective recesses in one or more of the interface surfaces.

Preferably, the main body is formed of plastic.

Conveniently, the main body is transmissive to the first imaging signal.

Advantageously, one or more of the plurality of markers is a different shape to one or more others of the plurality of markers.

Preferably, the markers are radio-opaque markers.

Conveniently, the markers are reflective to an ultrasound signal such that the markers can be identified in ultrasound images.

Advantageously, the guide-tube further comprises a telescopically extendible and retractable section of the main body.

Preferably, at least one of the plurality of markers is embedded in the tubular main body.

Conveniently, at least one of the plurality of markers is adhered to the tubular main body.

Advantageously, the two sections of the elongate tubular main body comprise two halves of the elongate tubular main body.

Another aspect of the present invention provides a method of forming a guide-tube comprising: providing a elongate tubular main body having a cavity configured to receive a needle or other slender instrument; and providing a plurality of markers disposed along a length of the tubular main body, the markers being opaque to a first imaging signal, such that the position and orientation of the guide-tube in a frame of reference can be substantially unambiguously determined by identifying the location of each of the plurality of markers in the frame of reference using the first imaging signal wherein providing a plurality of markers comprises embedding at least one of the plurality of markers in the tubular main body by: forming two halves of the tubular main body, the two halves having respective interface surfaces configured to be joined to each other to form the tubular main body; arranging one or more of the plurality of markers in respective recesses in one or more of the interface surfaces; and joining the two halves of the tubular main body together to form the tubular main body.

Advantageously, providing the main body comprises forming a main body of plastic.

Preferably, providing the main body comprises providing a main body which is transmissive to the first imaging signal.

Conveniently, providing a plurality of markers comprises providing one or more of the plurality of markers as marker of a different shape to one or more others of the plurality of markers.

Advantageously, providing a plurality of markers comprises providing radio-opaque markers.

Preferably, providing a plurality of markers comprises providing markers which are reflective to an ultrasound signal such that the markers can be identified in ultrasound images.

Conveniently, the method further comprises providing a telescopically extendible and retractable section of the main body.

Conveniently, the method further comprises the steps of: arranging one or more of the plurality of markers in a mould; and injecting a material into the mould to form the tubular main body, such that the one or more of the plurality of markers are embedded in the tubular main body.

Preferably, the two sections of the elongate tubular main body comprise two halves of the elongate tubular main body.

Preferably, the method further comprises: drilling one or more recesses in the main body; arranging one or more respective markers in the or each recess such that the or each marker is at least partially received by a respective recess; and applying a filler material to the main body to hold the or each marker in its respective recess.

Advantageously, providing a plurality of markers comprises adhering at least one of the plurality of markers to the tubular main body.

Preferably the method further comprises the steps of: drilling one or more recesses in the main body; and adhering one or more respective markers to the or each recess such that the or each marker is at least partially received by a respective recess.

Another aspect of the present invention provides a surgical apparatus comprising: a base unit; an arm coupled to the base unit, the arm comprising a first section and a second section which are moveable with respect to each other about a joint, the arm being configured to receive a guide-tube; a tracking unit coupled to the joint and configured to track movement of the first section of the arm with respect to the second section of the arm; and a processor configured to: receive intra-operative image data relating to the guide-tube and the anatomy of a patient from which an initial position and orientation of the guide-tube relative to the anatomy of the patient can be determined, the image data including image data relating to a target location within the patient, receive tracking information from the tracking unit, and track the position and orientation of the guide-tube based on the initial position and orientation and the tracking information such that the guide-tube may be aligned with respect to the target location within the patient using the tracked position and orientation of the guide-tube to allow the delivery of a surgical instrument to the target location.

Conveniently, the apparatus is an orthopaedic surgical apparatus.

Advantageously, the arm further comprises one or more further joints each with a respective tracking unit.

Preferably, the apparatus is configured to operate in a passive mode of operation in which the first section of the arm is manually movable with respect to the second section of the arm while the movement is tracked by the tracking unit and an active mode of operation in which movement of the first section of the arm is drivable by a drive unit while the movement is tracked by the tracking unit.

Conveniently, the apparatus further comprises a clutch mechanism for selecting the passive and active modes of operation, wherein the drive unit is disengaged on activation of the clutch mechanism.

Advantageously, the arm further comprises one or more further joints each with a respective tracking unit and each with a respective clutch mechanism.

Preferably, further comprises a lock associated with the joint, the lock being configured to prevent substantial movement or resist movement of the first section of the arm with respect to the second section of the arm.

Advantageously, the apparatus further comprises a processor configured to actuate the lock to prevent or resist movement of the first section of the arm with respect to the second section of the arm such that a guide-tube received by the arm is restrained from moving out of a desired movement path.

Conveniently, the apparatus further comprises a user input device configured to receive user input so that one or more of an entry point, a target and a path associated with the anatomy of a patient can be selected.

Advantageously, the apparatus further comprises a screen and the processor is configured to output display information to the screen representing an image of part of the anatomy of a patient and one or more of the entry point, the target or the path.

Preferably, the processor is further configured to receive data representing the entry point and target, and determine a path between the entry point and the target.

Conveniently, the user input device is configured to receive user input to drag-and-drop one or more of the entry point, target and path to a different location.

Preferably, the processor is further configured to output display information to the screen representing the actual determined location or trajectory or projected trajectory of the guide-tube superimposed on intra-operative image data.

Another aspect of the present invention provides a orthopaedic surgical apparatus comprising: a base unit; an arm coupled to the base unit, the arm comprising a first section and a second section which are moveable with respect to each other about a joint; a drive unit configured to drive movement of the first section with respect to the second section; a tracking unit coupled to the joint and configured to track movement of the first section of the arm with respect to the second section of the arm, the apparatus being configured to operate in a passive mode of operation in which the first section of the arm is manually movable with respect to the second section of the arm while the movement is tracked by the tracking unit and an active mode of operation in which movement of the first section with respect to the second section of the arm is drivable by the drive unit while the movement is tracked by the tracking unit.

Conveniently, the apparatus further comprises a processor configured to: receive intra-operative image data relating to a guide-tube received by the arm and the anatomy of a patient from which an initial position and orientation of the guide-tube relative to the anatomy of the patient can be determined, receive tracking information from the tracking unit, and track the position and orientation of the guide-tube based on the initial position and orientation and the tracking information.

Advantageously, the arm further comprises one or more further joints each with a respective tracking unit.

Preferably, the apparatus further comprises a clutch mechanism for selecting the passive and active modes of operation.

Conveniently, the arm further comprises one or more further joints each with a respective tracking unit and each with a respective clutch mechanism.

Advantageously, the apparatus further comprises a lock associated with the joint, the lock being configured to prevent substantial movement or resist movement of the first section of the arm with respect to the second section of the arm.

Conveniently, the apparatus further comprises a processor configured to actuate the lock to prevent or resist movement of the first section of the arm with respect to the second section of the arm such that a guide-tube received by the arm is restrained from moving out of a desired movement path.

Preferably, the apparatus further comprises a user input device configured to receive user input so that one or more of an entry point, a target or a path associated with the anatomy of a patient can be selected.

Conveniently, the apparatus further comprises a screen configured to display information representing an image of part of the anatomy of a patient and one or more of the entry point, the target or the path.

Advantageously, the processor is configured to receive data representing the entry point and target, and determine a path between the entry point and the target.

Preferably, the user input device is configured to receive user input to drag-and-drop one or more of the entry point, target and path to a different location.

Conveniently, the processor is further configured to output display information to the screen representing the actual determined location or trajectory or projected trajectory of a guide-tube superimposed on intra-operative image data.

Another aspect of the present invention provides a method of operating a surgical apparatus comprising: receiving intra-operative image data relating to a guide-tube and an anatomy of a patient, the surgical tool being received by an arm which is coupled to a base unit of the surgical apparatus, the image data including image data relating to a target location within the patient; determining an initial position and orientation of the guide-tube relative to the anatomy of the patient; receiving tracking information from a tracking unit which is coupled to a joint about which a first and a second section of the arm are moveable with respect to each other; and tracking the position and orientation of the guide-tube based on the initial position and orientation and the tracking information such that the guide-tube may be aligned with respect to the target location within the patient using the tracked position and orientation of the guide-tube to allow the delivery of a surgical instrument to the target location.

Another aspect of the present invention provides a method of operating an orthopaedic surgical apparatus comprising: activating a drive unit to drive movement of a first section of an arm with respect to a second section of an arm and tracking movement of the first section of the arm with respect to the second section of the arm about a joint, the arm being coupled to a base unit; and manually moving the first section of the arm with respect to the second section of the arm while the movement is tracked by the tracking unit.

Another aspect of the present invention provides an apparatus further comprising a disposable guide-tube.

Preferably, the apparatus further comprises an imaging device having an emitter and a receiver, the emitter being configured to emit the first imaging signal to the receiver which is configured to receive the first imaging signal, such that image data representative of the plurality of markers is obtainable from the imaging device by positioning the plurality of markers between the emitter and receiver.

Advantageously, the first imaging signal is an x-ray signal.

Preferably, the imaging device is a fluoroscope.

Another aspect of the present invention provides a method comprising: providing an arm coupled to a base unit of a surgical apparatus, the arm comprising a first section and a second section which are moveable with respect to each other about a joint, the arm being configured to receive a guide-tube; tracking movement of the first section of the arm with respect to the second section of the arm using a tracking unit coupled to the joint; and in a processor: receiving intra-operative image data relating to the guide-tube and the anatomy of a patient from which an initial position and orientation of the guide-tube relative to the anatomy of the patient can be determined, the image data including image data relating to a target location within the patient, receiving tracking information from the tracking unit, and tracking the position and orientation of the guide-tube based on the initial position and orientation and the tracking information such that the guide-tube may be aligned with respect to the target location within the patient using the tracked position and orientation of the guide-tube to allow the delivery of a surgical instrument to the target location.

Embodiments of the present invention will now be described, by way of example, with reference to the accompanying drawings in which:

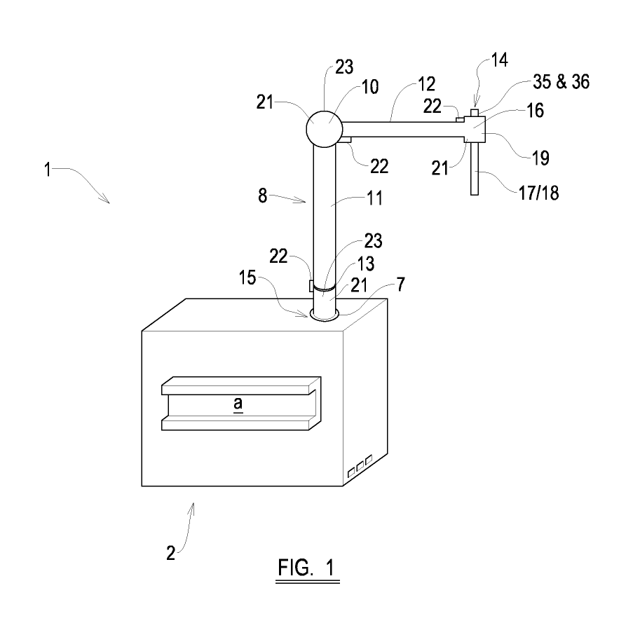

FIG. 1 shows aspects of a surgical apparatus in accordance with an embodiment of the present invention;

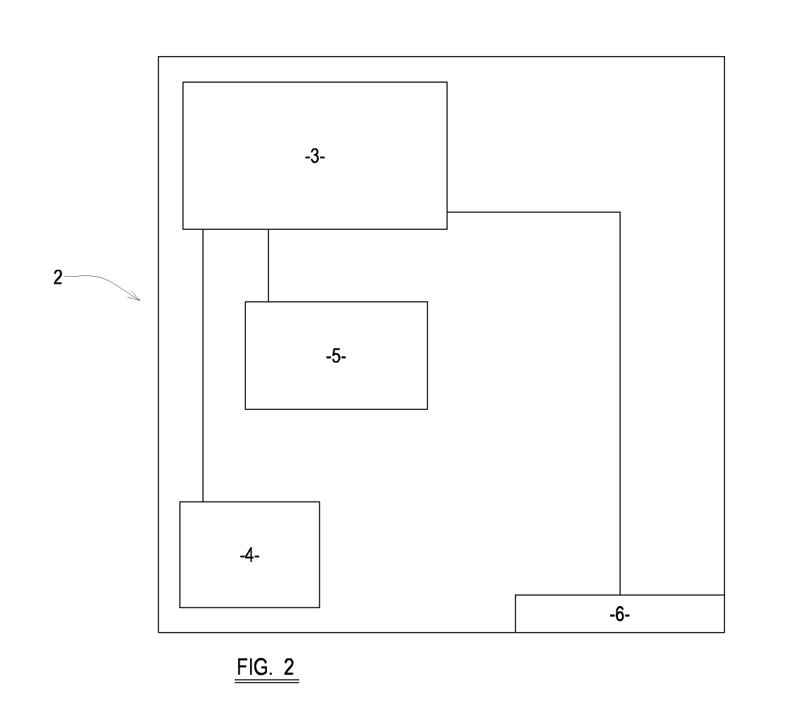

FIG. 2 shows a base unit in accordance with an embodiment of the present invention;

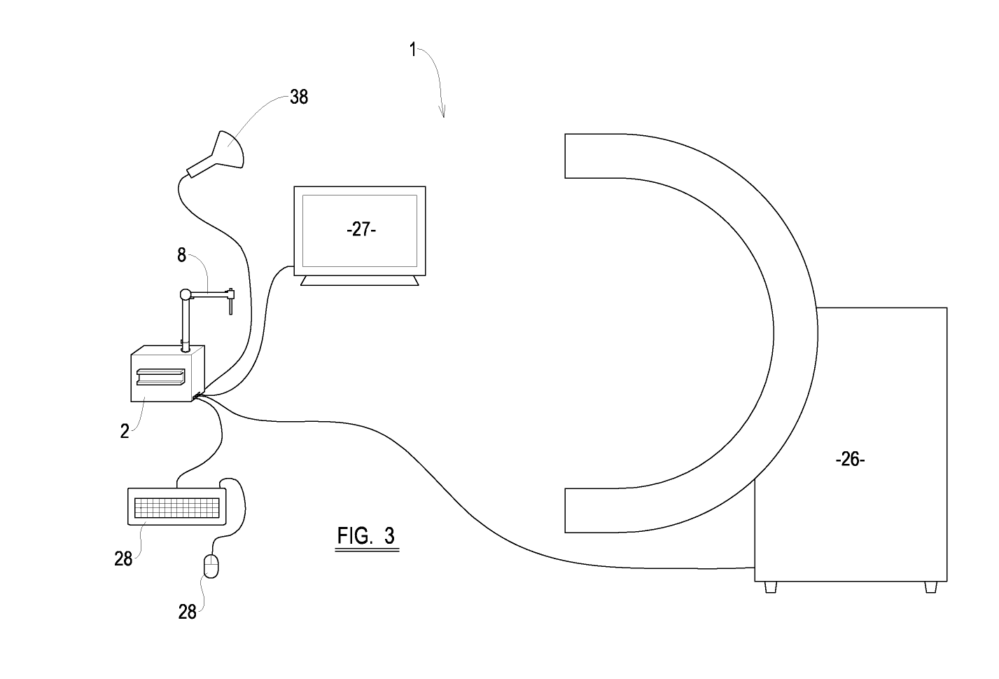

FIG. 3 shows aspects of a surgical apparatus in accordance with an embodiment of the present invention;

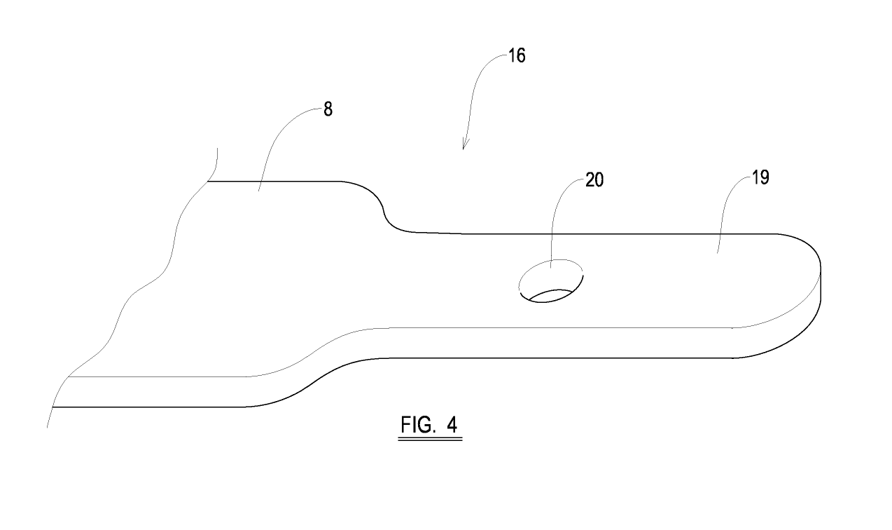

FIG. 4 shows aspects of a surgical apparatus in accordance with an embodiment of the present invention;

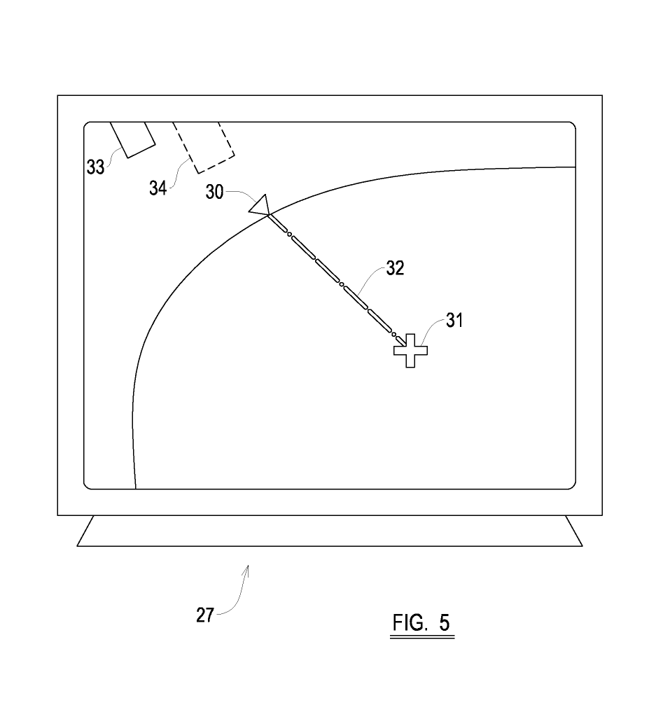

FIG. 5 shows aspects of a surgical apparatus in accordance with an embodiment of the present invention;

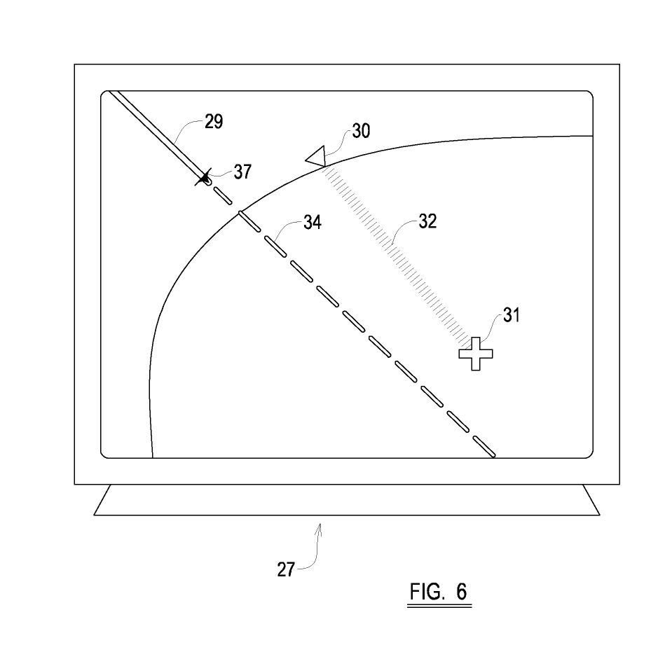

FIG. 6 shows aspects of a surgical apparatus in accordance with an embodiment of the present invention; and

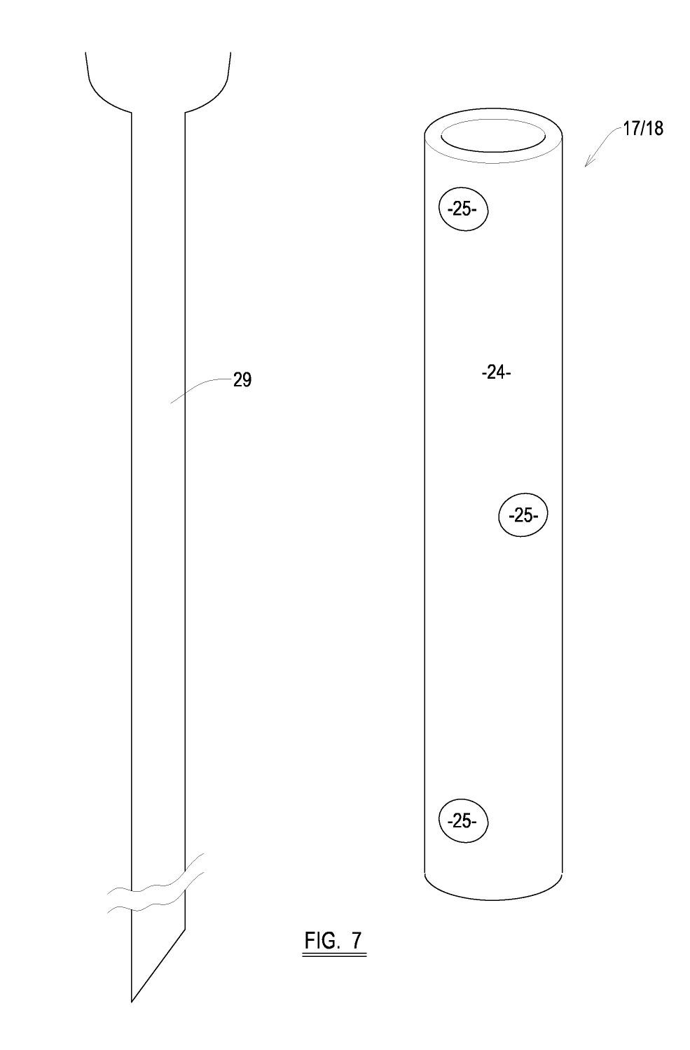

FIG. 7 shows aspects of a surgical apparatus in accordance with an embodiment of the present invention.

With reference to FIG. 1, an embodiment comprises a surgical apparatus 1. The surgical apparatus 1 comprises a base unit 2 and an arm 8.

Turning to FIG. 2 the base unit 2—in an embodiment—comprises a housing for a processor 3, control circuitry 4, tracking circuitry 5, one or more connectors 6, and an attachment location 7 for the arm 8—it will be appreciated that the housing may not contain all of these components and some embodiments of the housing include only some of these components.

The processor 3 is in electronic communication with the control circuitry 4, the tracking circuitry 5 and the one or more connectors 6. The interaction of the components of the base unit 2 will be more apparent following the discussion of the operation of the surgical apparatus 1 which is presented below.

The base unit 2 may include a clamp 9—see FIG. 1—configured to secure the base unit 2 to part of a bed or operating table (not shown). Preferably, the clamp 9 is adapted to secure the base unit 2 to a frame (not shown). The frame may be a rail of an operating table or bed.

In an embodiment, the base unit 2 includes a clamp 9 which is configured to secure the base unit 2 to part of a patient on which an operation is to be performed. In an embodiment, the base unit 2 includes a clamp 9 which is configured to secure the base unit 2 to a bone of the patient—this may be achieved by screwing a part of the clamp 9 into the bone of the patient. In an embodiment, the base unit 2 is configured to be strapped to a limb or other part of the patient.

In an embodiment, the base unit 2 includes a clamp 9 which is configured to secure the base unit 2 to a brace or clamp which holds part of the patient in a substantially fixed arrangement with respect to an operating table or bed.

In an embodiment, the base unit 2 includes a clamp 9 which is configured to secure the base unit 2 to an imaging device such as a fluoroscope, CT scanner, or MRI scanner. In this embodiment, the clamp 9 may be configured to secure the base unit 2 to an object (e.g. an arm or frame) which is, itself, secured to the imaging device rather than being configured to be secured directly to the imaging device.

An arm 8 is attached to the attachment location 7 of the base unit 2 and extends away from the base unit 2 substantially in a first direction. The attachment of the arm 8 to the base unit 2 may be a detachable attachment. Such that the arm 8 may be removed from the base unit 2 for storage and/or cleaning and the like.

In an embodiment, the arm 8 is secured to a manually adjustable gross-movement arm (not shown) and the manually adjustable gross-movement arm is attached to the attachment location 7. The manually adjustable gross-movement arm can be manually moved substantially freely and then locked in a fixed position with respect to the attachment location 7 and base unit 2.

In an embodiment, the arm 8 comprises at least one arm joint 10 such that the arm 7 may comprise a first section 11 and a second section 12 coupled to each other by an arm joint 10 in an articulated arrangement. Further arm joints 10 and arm sections may be provided in other embodiments. In an embodiment, the arm 8 is configured to rotate with respect to the base unit 2. As such, the arm 8 may be provided with a rotatable joint member 13 which allows rotation of the first section 11 of the arm 8 with respect to the base unit 2 about an axis of the rotatable joint member 13 which is preferably substantially parallel to the first direction (i.e. substantially parallel to the direction in which the arm 8 extends away from the base unit 2). Other arrangements of joints 10, 13 are also possible for the arm 8.

A proximal end 14 of the arm 8 is attached to the base unit 2 and a distal end 15 of the arm 8 is a free end of the arm 8. An attachment arrangement 16 is provided substantially at the distal end 15 of the arm 8.

The attachment arrangement 16 is configured to receive a guide-tube 18 such as a cannula 17. As such, the attachment arrangement 16 may comprise an extension 19 with an aperture 20 through an entire depth thereof. The aperture 20 is configured to receive the guide-tube 18 therethrough (see FIG. 4).

The attachment arrangement 16 may include a securing mechanism (not shown) to lock the guide-tube 18 in a fixed position and orientation with respect to the attachment arrangement 16.

In an embodiment, the attachment arrangement 16 includes a drive mechanism (not shown) to drive movement of the guide-tube 18 through the attachment arrangement 16 such that the guide-tube 18 can be advanced towards or retracted away from a patient. The drive mechanism preferably maintains the orientation of the guide-tube 18 with respect to the attachment arrangement 16 during operation thereof.

In an embodiment, the attachment arrangement 16 is configured to allow manual movement of the guide-tube 18 through the attachment arrangement 16 such that the guide-tube 18 can be advanced towards or retraced from a patient. The attachment arrangement 16 preferably maintains the orientation of the guide-tube 18 with respect to the attachment arrangement 16 during such manual movement of the guide-tube 18.

The surgical apparatus 1 includes one or more drive units 21. The or each drive unit 21 is configured to drive a movement of at least a section of the arm 8 with respect to another section of the arm 8 or the base unit 2—for example, a drive unit 21 may be provided to drive movement of the second section 12 of the arm 8 with respect to the first section 11 of the arm 8 about the arm joint 10. Another drive unit 21 may be provided to drive movement of the first section 11 of the arm 8 about the rotatable joint member 13 with respect to the base unit 2. The or each drive unit 21 may comprise one or more motors and one or more gears (not shown).

The or each drive unit 21 may be located in the base unit 2 or may be secured to the arm 8. In an embodiment, a plurality of drive units 21 are provided and each drive unit is in a different location in the surgical apparatus 1. Preferably, the or each drive unit 21 is located substantially adjacent the joint 10,13 about which that particular drive unit 21 is configured to drive movement of the arm 2—to reduce the length of any associated drive train. In an embodiment, the or each drive unit 21 is remote (and not adjacent) the joint 10,13 about which that particular drive unit 21 is configured to drive movement of the arm 2 (e.g. the or each drive unit 21 may be provided in the base unit 2 or in a drive unit casing (not shown) which is remote from the base unit 2).

The or each drive unit 21 may be under the automated control of the surgical apparatus 1 and, in particular, the control circuitry 4.

One or more locks 22 may be provided and configured to lock a joint 10,13 of the arm 2 so that movement of the arm 8 about that joint 10,13 is substantially prevented. The or each lock 22 may be manually operated (such that a user can manually lock the position and orientation of the arm 2 about the joint 10,13 by, for example, pressing a button) or may be under the automated control of the surgical apparatus 1. In particular, the control circuitry 4 housed in the base unit 2 may include elements to control automatically the activation (i.e. the locking) of the or each lock 22.

A lock 22 may be associated with the drive mechanism which is configured to drive movement of the guide tube 18 (if provided).

In an embodiment, a plurality of locks 22 are provided, two or more of the locks 22 may be coupled to a synchronous lock mechanism (not shown) which can be used manually or by the control circuitry 4 to lock the two or more locks 22 coupled to the mechanism substantially simultaneously.

In an embodiment, the or each lock 22 is configured to apply a braking or resistance force which, for example, resists movement of one section of the arm 8 with respect to another section of the arm 8 or which resists movement of the guide tube 18 with respect to the arm 8, or a section of the arm 8 with respect to the base unit 2. This braking or resistance force may be a range of forces—for example a low braking or resistance force may result in substantially free movement of the one section of the arm 8 with respect to another section of the arm 8 about a joint 10,13, and a high braking or resistance force may substantially prevent movement of one section of the arm 8 with respect to another section of the arm 8 about a joint 10,13.

The or each lock 22 may be operable such that movement of a substantial part of the arm 8 is resisted or substantially prevented but movement of a wrist (not shown) at the distal end 15 of the arm 8—such as between the distal end 15 of the arm 8 and the attachment arrangement 16—is not substantially prevented from movement or, in an embodiment, movement of the wrist is not restricted. Thus, the majority of the arm 8 can be positioned in the desired manner with respect to the patient and the wrist can be used to fine-tune the position and orientation of the guide-tube 18 with respect to the patient. This is an example of a fine adjustment mechanism (in this sense the arm 8 and joints 10,13 could be described as a coarse adjustment arrangement).

One or more tracking units 23 are provided. Preferably, one tracking unit 23 is provided for each joint 10,13 of the surgical apparatus 1. The or each tracking unit 23 is configured to track movement of a section of the arm 8 about a joint 10,13. A tracking unit 23 may also be provided to track the movement of the guide-tube 18 with respect to the arm 8.

In an embodiment, the or each tracking unit 23 comprises an optical tracking unit having an encoder wheel (for example encoded with grey code), a light emitter and light detectors—arranged in a conventional manner—such that the absolute position of the encoder wheel can be determined. Alternative tracking unit implementations are also possible.

The or each tracking unit 23 is in electronic communication with the tracking circuitry 5 of the base unit 2. Thus, position information regarding the position of each section of the arm 8 is returned to the tracking circuitry 5. The position of the arm 8 as a whole can, of course, be determined from the position of each section of the arm 8 relative to the adjacent section or sections of the arm 8 or base unit 2. The tracking unit 5 is configured to monitor the location of the attachment arrangement 16 of the surgical apparatus 1 with respect to the base unit 2.

One or more of the or each drive units 21 is provided, in an embodiment, with a clutch mechanism configured to engage or disengage a drive train of the drive unit 21 selectively such that one section of the arm 8 can be manually moved about a joint 10,13 with respect to another section of the arm 8 or base unit 2 without causing movement of the drive train (by actuating the associated clutch mechanism to disengage the drive train). It will be appreciated that a clutch mechanism may be associated with one or more joints 10,13 in this manner.

In an embodiment, the clutch mechanism is selectively actuated by a manual switch. Preferably, actuation of the switch engages the clutch mechanism and also at least partially disengages one or more of the one or more locks 22. Thus, actuation of the clutch mechanism may allow manual movement of at least section of the arm 8. The or each lock 22 may be disengaged so as to allow this movement but may still provide a braking or resistive force which resists further movement of one section of the arm 8 with respect to another section of the arm 8 or base unit 2 if an unpermitted manual movement is attempted—see below for more details regarding unpermitted manual movements.

When a clutch mechanism is actuated, the tracking unit 23 associated with the drive unit 21 for which the clutch mechanism is provided is not disengaged but continues to monitor the location of the attachment arrangement 16 of the surgical apparatus 1 with respect to the base unit 2.

A synchronous clutch actuation mechanism (not shown) may be provided. The actuation of the synchronous clutch actuation mechanism engages and disengages two or more clutch mechanisms of the surgical apparatus 1 substantially simultaneously. In an embodiment, the synchronous clutch actuation mechanism is coupled to the synchronous lock mechanism such that clutch mechanisms associated with a plurality of joints 10,13 can be engaged and the corresponding lock or locks 22 operated substantially simultaneously.

In an embodiment, the surgical apparatus 1 further comprises a disposable guide-tube 18 which may be a cannula 17 (see FIG. 7). The guide-tube 18 generally comprises a tubular main body 24.

The tubular main body 24 is formed from a radio-translucent or -transparent material. In other words, an x-ray image including the tubular main body 24 does not show the tubular main body 24 in a substantial manner and the tubular main body 24 does not substantially obscure other objects in an x-ray image. The material from which the tubular main body 24 is formed is substantially transmissive to x-rays.

The guide-tube 18 further comprises a plurality of markers 25. The markers 25 are opaque to an intra-operative imaging signal which may be a signal in any appropriate intra-operative imaging modality—for example, the imaging signal may be a radio-frequency signal (e.g. x-rays) or may be an ultrasound signal or the like. The markers 25 are, therefore, not transmissive to the imaging signal. Preferably, the markers 25 are radio-opaque markers 25—in other words, markers which are formed from a material which is generally not transmissive to x-rays—and the radio-opaque markers appear in an x-ray image. The markers 25 may or may not be optically opaque (in the visible light part of the radio-frequency spectrum). The markers 25 are preferably opaque to the extent that the boundary of each marker 25 can be detected using a particular imaging modality.

For the sake of convenience, embodiments of the present invention will be described with reference to “radio-opaque” markers 25 and “x-ray imaging” as the intra-operative imaging modality. However, it will be appreciated that the description is equally applicable to other imaging modalities and markers which are opaque to imaging signals of those modalities.

The plurality of markers 25 preferably comprise four or more markers 25. The plurality of markers 25 are arranged in a known pre-determined manner with respect to the tubular main body 24 such that a stereo-like x-ray image of the tubular main body 24 and markers 25, allows for the position and orientation of the tubular main body 24 to be determined (substantially unambiguously) in the frame of reference as the anatomy of the patient. The plurality of markers 25 are arranged along a length of the guide-tube 18.

One or more of the markers 25 may be a different shape to one or more of the other markers 25 such that one or more of the markers 25 is identifiable in an intra-operative image as different from another of the markers 25. It will be appreciated that this may allow registration to occur based on fewer images. Each of the markers 25 may be different from each of the other markers 25 such that each marker 25 may be uniquely identified from the other markers 25.

The stereo-like x-ray images preferably comprise a pair of images of the tubular main body 24 and markers 25 through two substantially orthogonal axes (these images need not be taken simultaneously). The x-ray images may be captured substantially simultaneously but preferably a single fluoroscope 26 (see FIG. 3) is used to capture both of the pair of images forming the stereo-like x-ray images; the fluoroscope 26 is moved between a first position in which a first x-ray image is captured and a second position in which the second x-ray image is captured—during this time the surgical apparatus 1 maintains the guide-tube 18 in a substantially fixed position and orientation with respect to the patient.

Registration could occur in numerous different manners and above is just one example. In another example of a registration process, an object of known dimensions is secured by a first end thereof in a fixed position with respect to the patient (for example, the object may be attached to a bone of the patient).

The first end of the object (which is preferably an elongate rod) is imaged using, for example, a fluoroscope. The first end of the object is secured to the patient and so the patient is also imaged as part of this process. A second end of the object (which opposes the first end thereof), is then imaged without imaging the patient. The second end is imaged a plurality of times through different imaging axes. The images of the second end of the object include images of the guide-tube 18. As the object has known dimensions the anatomy of the patient and the guide-tube 18 can be registered in the same frame of reference. This registration process allows registration with limited exposure of the patient to the imaging signal.

The surgical apparatus 1, in an embodiment, includes a fluoroscope 26.

In an embodiment, one of the one or more connectors 6 of the base unit 2 is configured to be connected to a fluoroscope 26. Image information from the fluoroscope 26 can be transmitted to the base unit 2 through the connector 6 where it may be processed by the processor 3. The processor 3 is operable to receive image data from a fluoroscope 26 and to identify the presence and position of radio-opaque markers 25 within the image data. The processor 3 may, in an embodiment, also be operable to identify one or more anatomical landmarks in the image data—these may be anatomical landmarks of a patient at least partially captured in the image data. In order to achieve these operations the processor 3 captures image frames from the fluoroscope 26 (i.e. the processor 3 acts as a frame grabber).

The surgical apparatus 1, in an embodiment, includes a screen 27. One of the one or more connectors 6 of the base unit 2 is preferably configured to be connected to the screen 27. The processor 6 is operable to output image data for display on the screen 27. This image data may include representations of fluoroscopic images which may be received through another of the connectors 6. The processor 6 is, in an embodiment, configured to overlay image data onto received fluoroscopic image data and to output this combined image data to the screen 27.

In an embodiment, an input device 28 is provided as part of the surgical apparatus 1. The input device 28 may comprise a computer mouse or keyboard or both.

In an embodiment, the input device 28 comprises the screen 27. In this embodiment, the screen 27 may be a touch sensitive screen. A user can enter information into the surgical apparatus 1 by touching the screen 27 with their finger or a stylus—as will be described below in more detail.

In an embodiment, the processor 3 does not automatically identify the markers 25. Instead, the representations of fluoroscopic images are displayed on the screen 27 and the user can manually identify the markers 25 in the images using the input device 28.

Methods of operation of the above described embodiments of the surgical apparatus 1 will now be described by way of example.

In accordance with an embodiment, a method is provided for aligning a guide-tube 18 with respect to a patient for use in the insertion of a needle 29 through the guide-tube 18 into the patient to a target location.

The patient is provided on a bed or operating table. The patient is preferably immobilised or the relevant part of the patient (i.e. the part on which the operation is to be performed) is immobilised. The base unit 2 of the surgical apparatus 1 is secured to the bed or operating table by the use of the clamp 9 or by some other mechanism. In other words, the base unit 2 is positioned in a substantially fixed location with respect to the patient.

The arm 8 may be detachable from the base unit 2 (as described above). If the arm 8 is a detachable arm 8, then the arm 8 is attached to the attachment location 7 of the base unit 2.

A guide-tube 18 is attached to the distal end (i.e. the free end) of the arm 8. The guide-tube 18 includes—as described above—a plurality of radio-opaque markers 25 along a length thereof.

A fluoroscope 26, which may or may not form part of the surgical apparatus 1, is provided. The fluoroscope 26 is connected to the base unit 2 and the base unit 2 is connected to a screen 27.

The arm 8 is positioned so that the guide-tube 18 is within the field of view of the fluoroscope 26 along with part of the anatomy of the patient (typically, the part of the anatomy of the patient is generally in the region on which the operation is to be performed).

The fluoroscope 26 is typically calibrated at this stage to reduce or eliminate inherent image distortion associated with this imaging modality. In particular, fluoroscopic images suffer from pin-cushion distortion. This distortion can be reduced or eliminated by using the fluoroscope 26 to image a grid of predetermined form (for example). An image transformation can then be calculated using fluoroscopic images of the grid and the known form of the grid. This transformation can then be applied to intra-operative images captured by the fluoroscope 26.

The fluoroscope 26 is activated to capture an image of the markers 25 of the guide-tube 18 and part of the anatomy of the patient. The fluoroscope 26 is then moved with respect to the patient and the guide-tube 18 and a second image of the markers 25 of the guide-tube 18 and part of the anatomy of the patient is captured from a different angle—the position of the guide-tube 18 with respect to the patient is not changed during this period. More images of the markers 25 of the guide-tube 18 and anatomy of the patient may be captured.

The captured images of the markers 25 of the guide-tube 18 and anatomy of the patient are delivered to the base unit 2. The processor 3 receives the captured images and identifies the markers 25 in the captured images (manual marker 25 identification is also possible—as discussed above). Using the captured images, the identified markers 25 in these images and the known arrangement of these markers 25, the processor 3 of the base unit 2 is able to register the location of the markers 25 with respect to the part of the anatomy of the patient captured in the images. As the markers 25 can be used to determine the position and orientation of the guide-tube 18, the registering of the markers 25 is also a registering of the position and orientation of the guide-tube 18 within a frame of reference of the surgical apparatus 1. The position of the part of the anatomy of the patient captured in the images is also registered in the frame of reference of the apparatus 1.

It will be appreciated that the guide-tube 18, the patient, and the fluoroscope 26 are arranged in a substantially fixed relationship with each other. Thus, a first pair of images captured by the fluoroscope 26 can be used to register the location of the guide-tube 18 in the frame of reference of the apparatus 1 and further images may be used to register the patient (or part thereof) in the frame of reference of the apparatus 1. The further images need not include the guide-tube 18. However, in a preferred embodiment, the guide-tube 18 and patient are registered in the frame of reference of the apparatus 1 using the same captured images.

The captured images comprise image data. This image data includes image data relating to the guide-tube 18 and the anatomy of the patient even if individual images which form part of the image data only include image data relating to one of the guide-tube 18 and the anatomy of the patient. Image data relating to an object or feature may be image data including a representation of that object or feature. In an embodiment, image data comprises on or more single representations each of which shows the anatomy of the patient or a part thereof and the guide-tube 18 or a part thereof.

The captured images are output by the processor to the screen 27.

The user uses the input device 28 to identify a target location in images captured by the fluoroscope 26 along with an entry point on or in the skin or bone of the patient (also in the images captured by the fluoroscope 26). The target location is a location within the patient which to which it is desired to deliver a surgical instrument such as a needle 29. The entry point on the skin of the patient is the location on the skin of the patient which the user has selected as the entry point for the surgical instrument (such as a needle 29).

Once the target location and entry point have been selected they may be identified in images displayed on the screen 17 with respective icons 30,31 (see FIGS. 5 and 6). This may be achieved by the processor 3 of the base unit 2 overlaying the icons 30,31 onto the fluoroscopic images.

The processor 3 of the base unit 2 may also overlay a projected path 32 between the entry point icon 30 and the target location icon 31. The user may review this projected path 32 to determine if the projected path 32 travels through or close to a part of the patient which is to be avoided (e.g. a particular organ, the outer portion of a bone or the like). In an embodiment, the user can use the input device 28 to drag-and-drop the entry point icon 30 and target location icon 31 to different positions within the images displayed on the screen 27. In an embodiment, the projected path 32 is continually re-calculated and re-displayed on the screen 27 during a drag-and-drop action such that the user can see the projected path 32 as the icons 30,31 are moved.

Once the entry point 30 and target location 31 icons have been placed on the desired entry point 30 and target location 31 as shown in the images displayed on the screen 27 the user may confirm the selected entry point and target location.

In an embodiment, only a path 32 (or trajectory) is identified by the user and displayed on the screen 27. This may be useful, for example, when the operation is the repair of a bone by the insertion of a pin from an end of a bone down a length of the bone.

In embodiments, any combination of entry point, target location and trajectory may be selected and the relevant icon or icons 30,31,32 displayed.

The arm 8 of the surgical apparatus 1 is then moved so that an end of the guide-tube 18 is positioned adjacent the entry point and the guide-tube 18 is substantially parallel with the projected path from the entry point to the target location—such that a substantially straight needle inserted through the tubular main body 24 of the guide-tube will generally pass through the entry point, travel along the projected path, and reach the target location.

The arm 8 of the surgical apparatus 1 may be moved manually—in a passive mode of operation. Alternatively, movement of the arm 8 of the surgical apparatus 1 may be driven by the or each drive unit 21 in an active mode of operating. In a given embodiment, the arm 8 may be operable in the passive mode, in the active mode, or selectively in either mode.

Tracking of the position of the guide-tube 18 after the initial registration process is achieved through the use of the or each tracking unit 23. Thus, the relatively complicated procedure of registering the location of the guide-tube 18 in the frame of reference of the surgical apparatus 1 need not be re-performed (i.e. it is a one-off registration procedure). The or each tracking unit 23 sends tracking data from the joints 10,13 of the arm 8 to the tracking circuitry 5 which determines the location of the guide-tube 18 within the frame of reference of the surgical apparatus 1. In other words, further intra-operative data need not be obtained in order to track the location of the guide-tube 18 with respect to the anatomy of the patient. In an embodiment, following the one-off registration procedure, the guide-tube 18 may be aligned with respect to the target location within the patient using only or solely the tracked position and orientation of the guide-tube 18 with respect to the patient anatomy to allow the delivery of a surgical instrument to the target location within the patient.

The control circuitry 4 may receive tracking information from the tracking circuitry 5 and may issue one or more signals to cause actuation of one or more of the locks 22 to restrain movement of the arm 8 in a manner which would position the guide-tube 18 or the arm 8 in an undesired location. For example, the or each lock 22 may be used to restrain and/or prevent movement of the arm 8 or guide-tube 18 into a volume which has been defined as a no-go volume (for example, in a region which is close to or comprising a part of a patient which is vulnerable to damage).

The or each lock 22 may be actuated to guide manual movement of the arm 8 in a desired manner (for example, such that the end of the guide-tube 18 is substantially parallel with the projected path from the entry point to the target and/or adjacent the entry point).

The or each lock 22 may restrain movement of the arm 8 as the arm 8 or guide-tube 18 approaches the boundary of a no-go volume or as it moves away from the desired line of movement and may substantially prevent movement of the arm 8 at the boundary or at a threshold distance from the desired line of movement.

The desired line of movement may be a movement path which has been determined by a user and input into the processor 3 (for example) or may have been determined by the processor 3 based on, for example, the initial position of the arm 8 and the entry point 30 and/or the target location 31 and/or the path 32. The movement path may be defined by a threshold distance from a line of desired movement and this threshold distance may decrease as the entry point is approached.

Thus, a user may be guided by use of the or each lock 22 during manual movement of the arm 8 and may be restrained and/or prevented from moving the arm 8 or guide-tube 18 into particular volumes (such as no-go volumes). The or each lock 22 may, in an embodiment, be independently actuated.

An icon 33 representing the actual position of the guide-tube 33 may be displayed on the screen 27. An icon 34 representing the desired location of the guide-tube 33 (based on the selected target location and entry point) may also be displayed on the screen 27. In an embodiment, the user can either confirm that the surgical apparatus 1 has correctly positioned with guide-tube 18 with respect to the patient by confirming the alignment of the icon 33 representing the actual position of the guide-tube 33 and the icon 34 representing the desired location of the guide-tube 33.

In an embodiment, an icon 34 is displayed on the screen 27 representing the actual projected trajectory of a substantially straight needle 29 through the guide-tube 34 (see FIG. 6). In this embodiment, a user may confirm correct alignment of the guide-tube 18 with respect to the patient by confirming that the icon 34 representing the actual projected trajectory passes through the target location icon 31 and the entry point icon 30 and/or along the desired path/trajectory 32.

In an embodiment, the or each lock 22 locks the arm 8 in the desired position following alignment of the guide-tube 18.

In an embodiment with a clutch mechanism, once the guide-tube 18 is in place, the user can actuate the clutch mechanism to allow for manual movement of at least one section of the arm 8 with respect to another section or the base unit 2. In an embodiment, the or each lock 22 limits the manual movement in this situation to permitted manual movement by applying a resistive or braking force to inhibit movement of one section of the arm 8 with respect to another section or the base unit 2. For example, the surgical apparatus 1 may be configured to permit small manual adjustments of the position of the guide-tube 18—movements beyond a certain range are restricted by the or each lock 22. This can be used as a safety mechanism to prevent injury to the patient.

In an embodiment, the arm 8 may be moved in a target centred arc such that the guide-tube 18 is always pointing towards the intended target (e.g. the entry point). In an embodiment, the manual movement of one section of the arm 8 with respect to another may cause one or more drive units 21 to drive movement of another section of the arm 8 with respect to a section of the arm 8 so as to maintain the guide-tube 18 in a predetermined configuration with respect to the patient—e.g. with the guide-tube 18 pointing towards the entry point).

In an embodiment, the or each lock 22 may permit movement of the guide-tube 18 (by movement of the arm 8) along a longitudinal axis of the guide-tube 18—thus, if a needle 29 has been inserted through the guide-tube 18 into the patient, movement in this manner is unlikely to cause tearing of tissue of the patient by movement of the needle 29 in a direction which is not parallel with a longitudinal axis of that needle 29. In an embodiment, the or each lock 22 restricts movement of the guide-tube 18 which would cause the guide-tube 18 to impact the patient (and potentially cause an injury). It will be appreciated that many different permitted and unpermitted movements of the guide-tube 18 can be configured. The permitted and unpermitted movements are preferably determined by the processor 3, the tracking circuitry 5, the control circuitry 4 or a combination thereof.

Once the guide-tube 18 is in the desired position with respect to the patient, a needle 29 can be inserted through the guide-tube 18 into the patient. That needle 29 will substantially follow the projected path 32. A user may, in an embodiment, manually insert the needle 29 into the patient through the guide-tube 18. In an embodiment a needle drive unit 35 may be provided. The needle drive unit 35 is preferably attached to the distal end 14 of the arm 8 and is configured to drive a needle 29 through the guide-tube 18.

A needle length tracker 36 may be provided which monitors the length of needle 29 which as passed through the guide-tube 18. As the guide-tube 18 is of a known length and has a known distance from the patient, data output by the needle length tracker 36 can be used to calculate the position of the needle within the patient. The needle length tracker 36 may be configured (perhaps in combination with the control circuitry 4 and processor 3) to issue a signal when the needle 29 has reached the target location. This signal may cause the actuation of an audio or visual notification (a visual notification may be displayed on the screen 27).

In an embodiment, the needle 29 is provided with a scale along its length so that the length of needle 29 which has been inserted into the patient can be determined by studying the scale.

In an embodiment, the guide-tube 18 is a telescopic guide-tube 18. The telescopic guide-tube 18 may be configured to allow only the desired length of needle 29 to be inserted into the patient in a number of different manners. For example, the telescopic guide-tube 18 may be telescopically moved (i.e. telescoped) towards a part of the anatomy of the patient until an extreme end of the tube abuts against the part of the anatomy of the patient. The length of telescopic deployment of the guide-tube 18 is recorded (automatically or manually) and this gives accurate information concerning the distance between the distal end of the guide tube 18 (in its telescopically retracted state) from the part of the anatomy of the patient. A similar effect may be achieved by moving a non-telescopic guide-tube 18 until a distal end of the guide-tube abuts against the part of the anatomy of the patient and then retracting the guide-tube 18 away from the patient and recording the distance of the movement of the guide-tube 18 (automatically or manually) away from the patient.

The processor 3 may overlay an icon 37 representing the position of the needle tip 37 onto fluoroscopic images displayed on the screen 27.

In an embodiment, the guide-tube 18 is positioned a pre-determined distance from the patient such that a needle 29 inserted through the guide-tube 18 can be inserted to approximately the desired depth such that the tip of the needle 29 is at the target location—e.g. because an end part of the needle 29 remote from the tip reaches or abuts against part of the guide-tube (or another part of the surgical apparatus 1) to prevent further movement of the needle 29 into the patient. Clearly, the distance between the tip of the needle 29 and the opposing end part of the needle 29 would need to be known to the surgical apparatus 1.

It will be appreciated that the needle 29 is just one example of a surgical instrument with which embodiments of the present invention may operate.

In an embodiment, a fluoroscope 26 is not used. Instead, the surgical apparatus 1 uses an ultrasound imaging device 38. In this embodiment, the ultrasound imaging device 38 is configured to be mounted to an attachment arrangement which has a known relationship with the attachment arrangement 16 for holding the guide-tube 18. The ultrasound imaging device 38 is used to obtain an image of a part of the anatomy of a patient and this image is displayed on the screen 27 as in the above described embodiments. In the case of this embodiment, however, registration of the guide-tube 18 and patient in the frame of reference of the surgical apparatus 1 is achieved by using the known relationship between the position of the ultrasound imaging device 38 and the guide-tube 18 and the known dimensions of the guide-tube 18—as will be apparent.

It will be understood that in this embodiment, the location of the ultra-sound imaging device 38 with respect to the guide-tube 18 must be known to a high degree of accuracy. As such, the ultra-sound imaging device 38 may be mounted to the surgical apparatus 1 by a mounting arrangement which is configured to hold the ultra-sound imaging device 38 in a predetermined location with respect to the guide-tube 18. The mounting arrangement may be keyed such that the ultra-sound imaging device 38 can only be attached to the arrangement in a predetermined orientation. The mounting arrangement may include one or more locking members to hold the ultra-sound imaging device 38 in position. In an embodiment, a calibration process is required to register the location of the ultra-sound imaging device 38 with respect to the guide-tube 18. For example, a calibration member may be provided which can be inserted through or mounted to the guide-tube 18 and which includes a section which can be identified by the ultra-sound imaging device 38—the location of the section with respect to the guide-tube 18 being known. Thus, the position of the guide-tube 18 with respect to the ultra-sound imaging device 38 can be determined. Alternatively, the guide-tube 18 may be configured such that it is visible within image data collected by the ultra-sound imaging device 38. The registration of the location of the guide-tube 18 with respect to the ultra-sound imaging device 38 is preferably a process which occurs once during a calibration step.

As will be appreciated, embodiments of the present invention can be used in combination with continual fluoroscopic or ultrasound imaging. Alternatively, embodiments of the present invention can use the initial fluoroscopic or ultrasound images captured during the registration process and not take continuous images of the progress of the operation. In this embodiment, periodic images can be acquired to check that the operation is proceeding as planned—e.g. at milestone events such as when the needle has reached the target location.

As will be understood, the target location is preferably a location within the patient which is not visible to the surgeon from the outside of the patient. In other words, the target location may be a visually obscured location to which a surgical instrument is to be delivered.

An embodiment of the invention comprises a method of manufacturing a disposable guide-tube 18 such as a cannula 17. The method generally comprises providing a mould for the guide-tube 18, positioning a plurality of markers 25 in predetermined locations within the mould, injecting a plastic material into the mould, and removing the needle-guide 18 from the mould.

In an embodiment the guide-tube 18 is moulded in two halves which are subsequently joined together. The faces of each half of the guide-tube 18 which are to be joined together in the finished guide-tube 18 (i.e. the interface surfaces) may, in this embodiment, be moulded to include recesses suitable to receive respective markers 25. Thus, markers 25 may be inserted into the recesses and the two halves of the guide-tube 18 joined together to retain the markers 25 in the recesses and to form the completed guide-tube 18. The recesses may be provided in one or more of the interface surfaces. The recesses may be drilled and not moulded.

Further steps of de-burring and the like may also be performed.

The plastic material which is injected into the mould is substantially transparent or translucent to an intra-operative imaging signal.

In another embodiment, the guide-tube 18 is formed and then recesses are drilled in the tubular main body 24. Markers 25 may then adhered to the tubular main body in the recesses. In an embodiment, markers 25 are placed in the recesses and then a material is used to fill the remainder of each recess. The filler material holds the markers 25 in place and may form a smooth outer surface of the guide-tube 18. The filler material may, therefore, form part of the tubular main body 24 of the guide-tube 18 and such markers 25 could be described as embedded in the tubular main body 24.

In an embodiment, the guide-tube 18 is formed by drilling a hole through a cylinder of material, drilling further holes (one for each marker) and inserting the markers 25 into the holes. The cylinder of material may, itself, be cut from a block of material.

In an embodiment, the guide-tube 18 is provided with a keyed attachment arrangement for attaching the guide-tube 18 to the attachment arrangement 16 in a known orientation.

It will be appreciated that although a needle 19 has been described above, embodiments are for use with other surgical instruments. The term “needle” is intended to encompass, inter alia, a hollow needle, a solid needle, a guide-wire, a screw, and the like, along with any surgical instrument which is suitable for insertion through a guide-tube 18. Indeed, it will be appreciated that embodiments of the present invention are suitable for use with a slender instrument other than a needle.

It will be appreciated that, in an embodiment, the processor 3, control circuitry 4, and tracking circuitry 5 may be combined in a single processor which may comprise various subcomponents (such as, a processor 3, control circuitry 4, and tracking circuitry 5). Indeed, any arrangement of features, devices, or processors may be provided to achieve the overall function of these elements 3,4,5; each feature, device or processor may be configured to perform all or part of the function of any of these elements 3,4,5.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. A surgical apparatus comprising:
a base unit;
an arm coupled to the base unit, the arm comprising a first section and a second section which are moveable with respect to each other about a joint, the arm being configured to receive a guide-tube;
a tracking unit coupled to the joint and configured to track movement of the first section of the arm with respect to the second section of the arm; and
a processor configured to:
receive intra-operative image data relating to the guide-tube and the anatomy of a patient from which an initial position and orientation of the guide-tube relative to the anatomy of the patient can be determined, the image data including image data relating to a target location within the patient,
receive tracking information from the tracking unit, and
track the position and orientation of the guide-tube based on the initial position and orientation and the tracking information such that the guide-tube may be aligned with respect to the target location within the patient using the tracked position and orientation of the guide-tube to allow the delivery of a surgical instrument to the target location without using further intra-operative image data to track the location of the guide-tube with respect to the anatomy of the patient.

2. An The apparatus according to claim 1, wherein the apparatus is an orthopaedic surgical apparatus.

3. (canceled)

4. The apparatus according to claim 1, wherein the apparatus is configured to operate in a passive mode of operation in which the first section of the arm is manually movable with respect to the second section of the arm while the movement is tracked by the tracking unit and an active mode of operation in which movement of the first section of the arm is drivable by a drive unit while the movement is tracked by the tracking unit.

5. The apparatus according to claim 4, further comprising a clutch mechanism for selecting the passive and active modes of operation, wherein the drive unit is disengaged on activation of the clutch mechanism.)

6. The apparatus according to claim 5, wherein the arm further comprises one or more further joints each with a respective tracking unit and each with a respective clutch mechanism.

7. The apparatus according to claim 1, further comprising a lock associated with the joint, the lock being configured to prevent substantial movement or resist movement of the first section of the arm with respect to the second section of the arm.

8. The apparatus according to claim 7, further comprising a processor configured to actuate the lock to prevent or resist movement of the first section of the arm with respect to the second section of the arm such that a guide-tube received by the arm is restrained from moving out of a desired movement path.

9. The apparatus according to claim 1 further comprising a user input device configured to receive user input so that one or more of an entry point, a target and a path associated with the anatomy of a patient can be selected.

10. The apparatus according to claim 9, further comprising a screen and the processor is configured to output display information to the screen representing an image of part of the anatomy of a patient and one or more of the entry point, the target or the path.

11. The apparatus according to claim 10, wherein the processor is further configured to receive data representing the entry point and target, and determine a path between the entry point and the target.

12. The apparatus according to claim 10 wherein the user input device is configured to receive user input to drag-and-drop one or more of the entry point, target and path to a different location.

13. The apparatus according to claim 10, wherein the processor is further configured to output display information to the screen representing the actual determined location or trajectory or projected trajectory of the guide-tube superimposed on intra-operative image data.

14. A method of operating a surgical apparatus comprising:
receiving intra-operative image data relating to a guide-tube and an anatomy of a patient, the guide-tube being received by an arm which is coupled to a base unit of the surgical apparatus, the image data including image data relating to a target location within the patient;
determining an initial position and orientation of the guide-tube relative to the anatomy of the patient;
receiving tracking information from a tracking unit which is coupled to a joint about which a first and a second section of the arm are moveable with respect to each other; and
tracking the position and orientation of the guide-tube based on the initial position and orientation and the tracking information such that the guide-tube may be aligned with respect to the target location within the patient using the tracked position and orientation of the guide-tube to allow the delivery of a surgical instrument to the target location without using further intra-operative image data to track the location of the guide-tube with respect to the anatomy of the patient.

15.-26. (canceled)

27. A guide-tube comprising:
an elongate tubular main body having a cavity configured to receive a needle or other slender instrument;
a plurality of markers disposed along a length of the tubular main body, the markers being opaque to a first imaging signal, such that the position and orientation of the guide-tube in a frame of reference can be substantially unambiguously determined by identifying the location of each of the plurality of markers in the frame of reference using the first imaging signal wherein the elongate tubular main body comprises two sections joined together at respective interface surfaces and one or more of the plurality of markers are arranged in respective recesses in one or more of the interface surfaces.

28. (canceled)

29. The guide-tube according to claim 27, wherein the main body is transmissive to the first imaging signal.

30. The guide-tube according to claim 27, wherein one or more of the plurality of markers is a different shape to one or more others of the plurality of markers.

31.-32. (canceled)

33. The guide-tube according to claim 27, further comprising a telescopically extendible and retractable section of the main body.

34. The guide-tube according to claim 27, wherein at least one of the plurality of markers is embedded in the tubular main body.

35.-37. (canceled)

38. The apparatus according to claim 1 further comprising a disposable guide-tube comprising:
an elongate tubular main body having a cavity configured to receive a needle or other slender instrument;
a plurality of markers disposed along a length of the tubular main body, the markers being opaque to a first imaging signal, such that the position and orientation of the guide-tube in a frame of reference can be substantially unambigulously determined by identifying the location of each of the plurality of markers in the frame of reference using the first imaging signal wherein the elongate tubular main body comprises two sections joined together at respective interface surfaces and one or more of the plurality of markers are arranged in respective recesses in one of more of the interface surfaces.

39.-45. (canceled)
